(19) United States
(12) Plant Patent Application Publication
(10) Pub. No.: US 2003/0028940 P1
(43) Pub. Date: Feb. 6, 2003

(54) Chrysanthemum plant named "Gedi One Oatl"

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,922

(22) Filed: Jun. 4, 2001

(51) Int. Cl. A01H 5/00

(52) U.S. Cl. PLT/295

(57) ABSTRACT

A new and distinct Chrysanthemum plant variety characterized by a daisy type inflorescence, consistent flowering response to short days, blooming consistently after 49 days of short day length, free branching habit, highly flexible plant habit, and very uniform round growth habit.

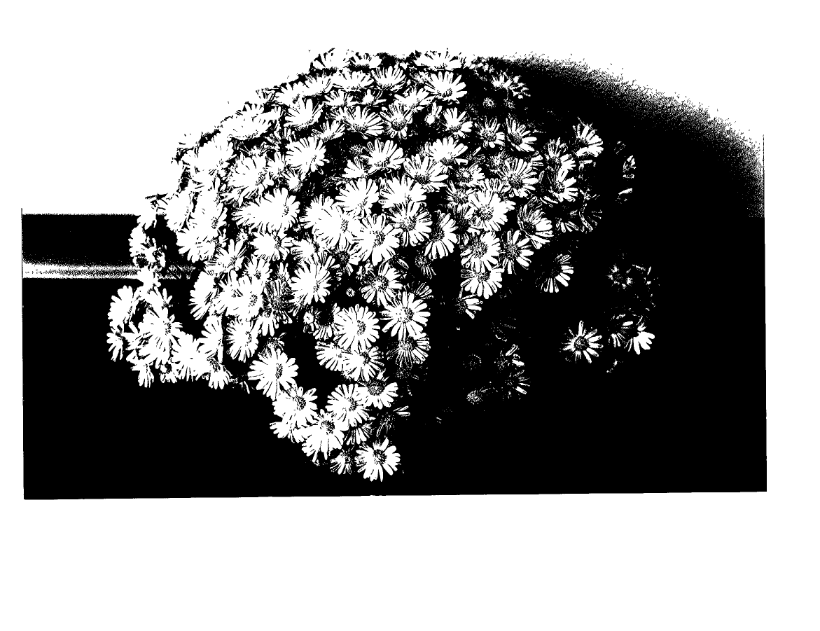

### BACKGROUND OF THE INVENTION

The present invention relates to a new and distinct cultivar of Chrysanthemum plant, botanically known as Dendranthema morifolium and herein after referred to by the cultivar name ‘GEDI ONE OATL.’

This new cultivar originated from a naturally occurring mutation on a single branch of the Chrysanthemum cultivar ‘Atlantico’ (unpatented in the United States). The new cultivar was discovered by the inventor in October 1998.

Asexual reproduction of the new cultivar by apical tip cutting and meristem tissue culture was performed in Oxnard, Calif. and has shown that the unique features of this new cultivar are stable and reproduced true to type on successive generations.

### SUMMARY OF THE INVENTION

The cultivar ‘GEDI ONE OATL’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE OATL.’ These characteristics in combination distinguish ‘GEDI ONE OATL’ as a new and distinct Chrysanthemum:

1. Daisy type inflorescence;

2. Consistent flowering response to short days, blooming consistently after 49 days of short day length;

3. Free branching habit;

4. Highly flexible plant habit; and

5. Very uniform round growth habit.

Plants of the new cultivar are similar to plants of the ‘Atlantico’ parent cultivar in most horticultural characteristics, however plants of the new cultivar differed from plants of the ‘Atlantico’ cultivar in ray floret color.

The new cultivar can be compared to the Chrysanthemum cultivar ‘Oriole’ (disclosed in U.S. Plant Pat. No. 10,181). However plants of the new Chrysanthemum differed from the plants of the cultivar ‘Oriole’ in the following characteristics:

1. The new cultivar naturally blooms approximately 2 weeks earlier;

2. The new cultivar has a more round plant shape and greater branching; and

3. The new cultivar has more blooms per flowering branch.

### BRIEF DESCRIPTION OF THE DRAWINGS

The photograph shows a plant of ‘GEDI ONE OATL.’ grown in a 1-gallon container. A single cutting was grown in the container and planted at the end of June. The plants were grown under outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial filed on Oxnard, Calif. in the month of September 2000. The growing conditions range from 60° to 65° F. at night to 70° to 85° during the day. Measurements and numerical values represent averages of typical flowering types.

Botanical classification: Dendranthema morifolium cultivar ‘GEDI ONE OATL’

Commercial classification: Garden-type Chrysanthemum

Parentage: Naturally occurring mutation on a single branch of the Chrysanthemum cultivar ‘Atlantico’ (unpatented in the United States)

### Propagation

Time to Rooting: 7 to 14 days at approximately 21° C.

Rooting habit: Fine, fibrous

### Plant

Growth Habit: Mounding herbaceous perennial

Height: Approximately 24.5 cm

Growth rate: Moderate

Branching Characteristics: Free Branching

Length of Lateral Branches: Approximately 21 cm

Number of leaves per lateral branch: Approximately 19

### Foliage

Leaf:

Arrangement.—Alternate Single

Average Length.—Approximately 2.6 cm

Average Width.—Approximately 1.9 cm

Shape of blade.—Ovate

Apex.—Acuminate

Base.—Attenuate

Attachment.—Stalked

Margin.—Serrate

Texture of top surface.—Lightly pubescent

Texture of bottom surface.—Lightly pubescent

Color.—young foliage upper side: Near 147A young foliage under side: Near 147B mature foliage upper side: Near 147A mature foliage under side: Near 147B venation upper side: Near 147B venation underside: Near 147B

Petiole:

Average Length.—Approximately 1.5 cm

### Bloom

Inflorescence:

Flowering habit.—Induced by darkness period greater than 13.5 hours, approximately 49 days of appropriate day length required to induce and develop blooms

Inflorescence form.—Daisy

Natural flowering season.—Approximately the last week of August, first week of September

Number of inflorescences per stem.—Approximately 13 to 15

Inflorescence diameter.—Approximately 3.4 cm

Inflorescence depth.—Approximately 0.9 cm

Inflorescence longevity on plant.—Approximately 26-29 days

Persistence.—Persistent

Ray Florets:

Appearance.—Matte

Texture.—Smooth

Average Number per flower.—Approximately 25 to 30

Shape.—Cylindric

Margin.—Entire

Apex.—Obtuse

Length.—Approximately 1.4 cm

Width.—Approximately 0.4 cm

Color.—upper surface at first opening: Near 178B upper surface at maturity: on 40% nearest to base: Near 7B upper surface at maturity: on 60% nearest to apex: Near 166B upper surface atfading: on 20% nearest to base: Near 7B upper surface at fading: on 80% nearest to apex: Near 166B under surface atfirst opening: Near 178B under surface at maturity: on 40% nearest to base: Near 7B under surface at maturity: on 60% nearest to apex: Near 166B under surface at fading: on 20% nearest to base: Near 7B under surface at fading: on 80% nearest to apex: Near 166B

Disc florets:

Appearance.—Shiny

Texture.—Smooth

Average number per flower.—Approximately 90 to 100

Shape.—Cylindric

Apex.—Obtuse

Average length.—Approximately 0.3 cm

Average width.—Approximately 0.1 cm

Color.—at first opening: Near 15A at maturity:Near 15A at fading: Near 15A

Peduncle:

Length.—at terminal end (shortest): Approximately 5 cm at lateral end (longest): Approximately 6.7 cm

Strength.—Moderate

Color.—Near 146A

Habit.—Erect

Inflorescence Bud:

Length.—Approximately 0.4 cm

Diameter.—Approximately 0.4 cm

Form.—Globular

Color.—Near 146A

Sepals:

Appearance.—Matte

Texture.—Lightly pubescent

Number.—Approximately 15 to 20

Shape.—Ovate

Margin.—Entire

Apex.—Acute

Color.—upper side: Near 137B under side: Near 137B

### Reproductive Organs

Ray Florets:

Pistillate.—

Number of pistils per flower.—1

Stigma shape.—2 branched

Stigma color.—Near 15A

Stigma length.—Approximately 0.1 cm

Style color.—Near 144B

Style length.—Approximately 0.3 cm

Disc Florets:

Pistillate.—Number of pistils per flower: 1 Stigma shape: Cylindric Style Length: 1.0 cm

Staminate.—Number of stamens per flower: Approximately 5 Anther shape: Tubular Anther color: Near 15A Pollen color: Near 15A

### Other Characteristics

Disease Resistance: Neither resistance nor susceptibility observed

What is claimed is:

1. A new and distinct variety of Chrysanthemum plant named ‘GEDI ONE OATL’ as herein illustrated and described.
